(19) United States
(12) Patent Application Publication
(10) Pub. No.: US 2006/0110824 A1
(43) Pub. Date: May 25, 2006

(54) Method for culturing avian primordial germ cells (PGCs) for a long period and preparing a medium for culturing avian PGCs for a long period

(76) Inventors: Lih-Ren Chen, Tainan (TW); Jui-Jane Tailiu, Tainan (TW); Chein Tai, Tainan (TW); Huan-Ting Lu, Tainan (TW)

Correspondence Address: DELLETT & WALTERS, P. O. BOX 82788, PORTLAND, OR, 97282-0788, US

(21) Appl. No.: 10/997,569

(22) Filed: Nov. 24, 2004

(51) Int. Cl. C12N 5/06

(52) U.S. Cl. 435/349

(57) ABSTRACT

The present invention is a method for culturing avian PGCs comprising providing fibroblast cells, culturing fibroblast cells in a basal medium, collecting a conditioned medium, providing avain gonadal cells with PGCs from sex glands, and growing the PGCs in the conditioned medium supplemented with growth factors, wherein the conditioned medium provides the avian PGCs being culturing for a long period. Furthermore, the present invention is a method for preparing a medium for culturing avian PGCs comprising providing a basal medium, culturing fibroblast cells in the basal medium, collecting a conditioned medium; and adding growth factors into the conditioned medium, wherein the conditioned medium provides the avian PGCs being cultured for a long period. The present invention is the conditioned medium prepared by the above method.

### BACKGROUND OF THE INVENTION

1. Field of the Invention

The present invention relates to a method for culturing avian primordial germ cells (PGCs), and more particularly to a method for culturing avian PGCs for a long period and a method for preparing a medium for culturing avian PGCs for a long period.

2. Description of Related Art

In recent years, stem cells have been used widely in clinical treatment and basic research about embryonic development. In the biotechnology field, stem cells were used in cloning to produce pharmaceutical human proteins at a high price.

Fowl and livestock have been used to manufacture pharmaceutical proteins, and the manufacturing methods continue to be important studies in biotechnology. The manufacturing methods were expected to have advantages of wide application, high economic benefits and low pollution. For instance, chickens produce 250 eggs every year so chickens can produce recombination proteins with high efficiency. Using chickens to produce human proteins can increase the efficiency and decrease the primary cost factor.

In fowl, conventional methods for cloning include microinjecting DNA into a zygote; transplanting PGCs or blastodermal cells into blood in an embryo of a domestic animal; or using sperms as a vector for transforming.

There were methods using PGCs with cloned genes to produce transgenic chimera chickens, such that transgenic chimera chickens with germline chimera could be obtained. The progenies of transgenic chimera chickens had foreign genes. Conventional methods to manufacture transgenic chimera chickens included separating PGCs from the sex glands, followed by transforming a desired foreign gene into the PGCs. Finally, the PGCs were transplanted into a chicken embryo to form a transgenic chimera chicken. However, in those conventional methods, PGCs from the sex glands had to be separated again and again. Thus, the PGCs could not be cultured for a long period so that the cell number is less and the cell origin is unstable. Furthermore, the conventional methods are complicated as PGCs had to be separated from stroma cells in the sex glands. By using the conventional methods, practitioners needed feeder cells to co-culture with PGCs and the PGCs could only be cultured for a short period.

### SUMMARY OF THE INVENTION

An aspect of the present invention relates to a method for culturing avian PGCs. The method comprises providing fibroblast cells; culturing fibroblast cells in a basal medium; collecting a conditioned medium; providing avian gonadal cells containing PGCs from sex glands; and growing the PGCs in the conditioned medium supplemented with growth factors, wherein the conditioned medium provides the avian PGCs being culturing for a long period.

Preferably, the avian PGCs are fowl PGCs. More preferably, the fowl PGCs are chicken PGCs.

Preferably, the fibroblast cells are inactivated or non-inactivated. More preferably, the inactivated fibroblast cells are inactivated with mitomycin C.

More preferably, the fibroblast cells are chicken embryonic fibroblast cells or mouse embryonic fibroblast cells. Most preferably, the mouse embryonic fibroblast cells are mouse STO fibroblast cells.

Preferably, the growth factors are leukocyte inhibition factor (LIF), stem cell factor (SCF), fibroblast growth factor, insulin growth factor-1, interleukin-11 or any combination of the foregoing factors.

Preferably, the method further comprises treating the gonadal cells with an enzyme to form a suspension. More preferably, the enzyme is trypsin.

Preferably, the suspension comprises PGCs and stroma cells.

Preferably, the suspension is further treated by steps comprising centrifuging the suspension to remove the supernatant; and resuspending the PGCs and stroma cells in the conditioned medium supplemented with the growth factor.

Preferably, the basal medium is Dulbecco's modified Eagle's medium (DMEM).

Another aspect of the present invention is to provide a method for preparing a medium for culturing avian PGCs. The method comprises providing a basal medium; culturing fibroblast cells in the basal medium; collecting a conditioned medium; and adding growth factors into the conditioned medium, wherein the conditioned medium provides the avian PGCs being culturing for a long period.

Preferably, the fibroblast cells are inactivated or non-inactivated. More preferably, the fibroblast cells are chicken embryonic fibroblast cells or mouse embryonic fibroblast cells. Most preferably, the mouse embryonic fibroblast cells are mouse STO fibroblast cells.

Preferably, the inactivated fibroblast cells are inactivated with mitomycin C.

Preferably, the growth factors are leukocyte inhibition factor (LIF), stem cell factor (SCF), fibroblast growth factor, insulin growth factor-1, interleukin-11 or any combination of the foregoing factors.

Preferably, the basal medium is DMEM.

Still another aspect of the present invention is a medium prepared by the foregoing method.

Further benefits and advantages of the present invention will become apparent after a careful reading of the detailed description.

### DETAILED DESCRIPTION OF THE INVENTION

In an aspect, the present invention relates to culturing chicken PGCs in vitro to amplify the amount of the PGCs, such that the chicken PGCs have a single origin and the transgenic efficiency is increased. The method according to the present invention can be employed to culture the PGCs in vitro for a long period. According to the present invention, the stability of the expressing of the foreign gene in the cloned cell increases, and the production efficiency of the transgenic fowl also increases.

Furthermore, the transgenic technique in fowl according to the present invention has developed very well. The superiority of fowls as a source of eggs makes the fowls excellent candidates in the application of transgenic techniques. The long period culturing of PGCs operated on with transgenic techniques applies to fowl gene cloning and providing large amounts of a desired cloned strain of fowl.

Specifically, a method for culturing avian PGCs according to the present invention comprises providing fibroblast cells; culturing fibroblast cells in a basal medium; collecting a conditioned medium; providing avian gonadal cells with PGCs from sex glands; and growing the PGCs in the conditioned medium supplemented with growth factors, wherein the conditioned medium provides the avian PGCs being culturing for a long period.

Preferably, the avian PGCs are fowl PGCs, and more preferably, the fowl PGCs are chicken PGCs.

In a preferred embodiment of the present invention, the fibroblast cells are inactivated or non-inactivated. The inactivated fibroblast cell may be inactivated with mitomycin C. Furthermore, the fibroblast cells preferably are chicken embryonic fibroblast cells or mouse embryonic fibroblast cells. More preferably, the mouse embryonic fibroblasts are mouse STO fibroblast cells.

In a preferred embodiment of the present invention, the growth factors may be a leukocyte inhibition factor (LIF), a stem cell factor (SCF), a fibroblast growth factor, an insulin growth factor-1, an interleukin-11 or any combination of the foregoing factors. The growth factors maintain growth of the PGCs.

Preferably, the basal medium for culturing the gonadal cells is Dulbecco's modified Eagle's medium (DMEM).

A preferred embodiment of the method for culturing PGCs according to the present invention further comprises treating the gonadal cells with an enzyme to form a suspension before growing the PGCs in the conditioned medium.

Preferably, the suspension comprises PGCs and stromas cells. More preferably, the enzyme is trypsin.

The method further comprises centrifuging the suspension to remove the supernatant. More preferably, the PGCs and the stroma cells are resuspended in the conditioned medium supplemented with the growth factors.

Specifically, a method for preparing a medium for culturing avian PGCs according to the present invention comprises providing a basal medium; culturing fibroblast cells in the basal medium; collecting a conditioned medium; and adding growth factors into the conditioned medium, wherein the conditioned medium provides the avian PGCs being culturing for a long period.

In a preferred embodiment of the present invention, the fibroblast cells are inactivated or non-inactivated. The inactivated fibroblast cells may be activated with mitomycin C. Furthermore, the fibroblast cells preferably are chicken embryonic fibroblast cells or mouse embryonic fibroblast cells.

In a preferred embodiment of the present invention, the growth factors may be leukocyte inhibition factor (LIF), stem cell factor (SCF), fibroblast growth factor, insulin growth factor-1, interleukin-11 or any combination of the foregoing growth factors. The growth factors can maintain the PGCs growth. A preferred basal medium is DMEM.

A culturing medium according to the present invention for avian PGCs is manufactured by the forgoing method.

While the following terms are believed to be well understood by one of ordinary skill in the art, the following definitions are set forth to any ambiguity in the explanation of the invention.

The term “avian” as used herein refers to any bird species, including but not limited to Gallinacea sp., chicken, turkey, duck, goose, quail and pheasant. The chicken is currently preferred.

The term “primordial germ cells (PGCs)” as used herein refers to cells derived from sex glands and exhibited an embryonic stem cell phenotype.

The term “basal medium” as used herein refers to any material suitable for culturing cells. For example, a preferred medium is DMEM and more preferably, the medium further contains FBS, chicken serum, sodium pyruvate, L-glutamine, 2-mercaptoethanol, streptomycin and penicillin.

The term “conditioned medium” as used herein refers to a growth medium that is further supplemented by factors derived from media obtained from cultures of fibroblast cell on which primordial germ cells can be cultured.

The term “STO cell” as used herein refers to embryonic fibroblast mouse cell such as are commercially available.

In a preferred embodiment of the present invention, a sex gland is taken from a 5.5 day chicken embryo. The sex gland is treated with a 0.05% solution of trypsin containing 0.53 mM EDTA to separate the gonadal cells. At this time, the PGCs must not separate from the stroma cells within the sex gland. The gonadal cells are culture in 4-well plates that are pretreated with 0.1% gelatin and grow in a conditioned medium that is cultured with inactivated/non-inactivated chicken embryonic fibroblast (CEF) or inactivated/non-inactivated mouse embryonic fibroblast (STO). Mouse leukemia inhibitory factor (mLIF), human stem cell factor (hSCF), basic fibroblast growth factor (bFGF), human insulin-like growth factor-1 (hIGF-1) and human interlukin-11 (hIL-11) are added to the conditioned medium. Experiments were conducted that provided data and results that show that all conditioned medium can be used to increase the development of the PGCs and form cell colonies. The stroma cells will not form cell colonies and can be easily washed out. The growth cells are stained by PGC specific nucleophilic dye and marked with an undifferentiated embryonic stem cell specific antibody. The data shows that the present method not only can sustain the growth and differentiation ability of PGCs but also can sustain the original character of the PGCs.

Thus, an aspect of the present invention pertains to the production of undifferentiated avian cells expressing an embryonic stem cell phenotype from avian gonadal cells comprising primordial germ cells collected from an avian embryo. The general view among those of ordinary skill in the art is that avian embryonic gonadal cells comprising primordial germ cells, such as may be collected from, for example, the avian embryonic genital ridge or sex glands when the embryo had reached a stage associated with gonadal development terminally differentiate to germ cells only. Hence, the process of the present invention provides for convenience during the culturing of undifferentiated cells expressing an embryonic stem cell phenotype.

The medium used in carrying out the present invention may be any suitable medium. The medium may be a conditioned medium or a synthetic medium, both of which are known in the art. Optionally, the medium may be supplemented with growth factors, including but not limited to leukemia inhibitory factor (LIF), insulin-like growth factor (IGF), fibroblast growth factor (FGF), basic fibroblast growth factor (bFGF) and stem cell factor (SCF—also called steel factor or SF).

The undifferentiated avian cells produced by the process of the present invention are useful, among other things, as a tool for the study of embryological development and the production of transgenic poultry. They are useful in allowing the application of homologous recombination to the production of transgenic poultry.

The undifferentiated avian cells expressing an embryonic stem cell phenotype should also be useful for the production of chimeric avian species including transgenic chimeric avian species. Transgenic chimeric avian species will be useful for the recovery of recombination proteins that preferably can be recovered directly from the eggs of such chimeric transgenic avian species. For example, such avian species can be used for the production and recovery of therapeutic proteins and other polypeptides.

Therefore, the advantages of the method according to the present invention include the following.

(1) The method does not need a feeder matrix to culture the PGCs.

(2) The method does not need to separate the PGCs from the stroma cells in the sex glands.

(3) The present invention uses a conditioned medium for culturing PGCs and sustains the original PGC's characteristics. The method is operated conveniently and decreasing cell injuries occurred in separating step. Further details of this invention are illustrated in the following examples.

### EXAMPLE

### Example 1

### Preparation of Conditioned Medium

1.1. Cell

non-inactivated chicken embryonic fibroblast (CEF)

inactivated CEF

non-inactivated STO

inactivated STO

1.2. Basal medium

DMEM culture medium containing 10% FBS, 2% chicken serum, 1 mM sodium pyruvate, 2 mM L-glutamine, 5.5×10⁻⁵ M 2-mercaptoethanol, 100 μg/ml streptomycin and 100 unit/ml penicillin.

1.3. Process for preparing conditioned medium by using non-inactivated STO and CEF

(1) Coating 0.1% gelatin on a 100×10 mm petri dish and taking 10 ml (5×10⁵ cells) of cell suspension cultured on the petri dish.

(2) After cell growth filled the dish, the original medium was removed and changed to 10 ml of a new basal medium.

(3) The used medium was collected every 24 hours for 7 to 10 days and changed to a new basal medium.

(4) The collected medium was centrifuged (200 g) for 5 minutes, and filtered with a 0.22 μm filter and stored at −20° C.

1.4. Process for preparing a conditioned medium by using inactivated STO and CEF

(1) Coating 0.1% gelatin on a 100×10 mm petri dish and taking 10 ml (5×10⁵ cells) of cell suspension cultured on the petri dish.

(2) After cell growth filled the dish, the original medium was removed and changed to 10 ml of a new basal medium containing mitomycin C for 2.5 hours.

(3) Removing the medium containing mitomycin C, washing the cells with serum-free PBS three times and culturing the cells in 10 ml of basal medium.

(4) The used medium was collecting every 24 hours for 7 to 10 days and changed to a new basal medium.

(5) The collected medium was centrifuged (200 g) for 5 minutes, and filtered with a 0.22 μm filter and stored at −20° C.

### Example 2

### Culturing the Chicken PGCs

2.1. Process

(1) A sex gland was isolated from a 5.5 day chicken embryo. Then, the sex gland was treated a 0.05% trypsin solution scontaining 0.53 mM EDTA to separate the gonadal cells. At this time, the PGCs were not separated from the stroma cells.

(2) 4-well plates were pretreated with 0.1% gelatin.

(3) The gonadal cell suspension was centrifuged (200 g) for 5 minutes, the supernatant was removed and resuspended with four different conditioned media that contained growth factors. The growth factors were 1000 unit/ml mLIF, 5 ng/ml hSCF, 10 ng/ml bFGF, 10 ng/ml hIGF-1 and 0.04 ng/ml hIL-11.

(4) The cell suspension was cultured on 4-well plates pretreated with 0.1% gelatin.

(5) The chicken PGCs were grown to form cell colonies for about 7 to 10 days.

(6) Half of each medium was changed every day, and the PGC were subcultured every two weeks.

2.2. Results

Table 1 shows the cell growth numbers of chicken PGCs cultured in four conditioned media.

TABLE 1

| | | Generation | | Cell |
|---|---|---|---|---|
| | Original | of | | number |
| | cell | Sub- | Culture | (colony |
| Medium | number | culture | Days | per well)* |
| Prepared by | 1.5 × 10³ | 4 | 82 | 796 |
| nactivated STO | | | | |
| cell | | | | |
| Prepared by | 1.5 × 10³ | 3 | 82 | 421 |
| non-inactivated | | | | |
| STO cell | | | | |
| Prepared by | 1.3 × 10³ | 4 | 103 | 860 |
| inactivated | | | | |
| CEF cell | | | | |
| Prepared by | 8 × 10³ | 1 | 35 | 356 |
| non-inactivated | | | | |
| CEF cell | | | | |

*Cell number in each colony greater than 10 were counted.

The results show that all conditioned medium increase the PGCs' growth and formed cell colonies. Furthermore, the PGCs maintained the ability to proliferate.

### Example 3

### Test of the Characteristics of the Cultured Cells

The cultured chicken PGCs prepared in example 2 were stained by PGC specific dye, which is periodic acid-Schiff (PAS) stain. Alternatively, the cultured chicken PGCs were stained with an undifferentiated embryonic stem cell marker, such as an anti-SSEA-1 antibody. If the cells were stained by the PGC specific nucleophilic dye or undifferentiated embryonic stem cell marker, the cells were determined to be PGCs and were not differentiated.

The results show that the cells prepared in example 2 maintained the capability to grow and differentiate and the original characteristics.

Although the invention has been explained in relation to its preferred embodiment, many other possible modifications and variations can be made without departing from the spirit and scope of the invention as hereinafter claimed.

What is claimed is:

1. A method for culturing avian PGCs comprising:
providing fibroblast cells;
culturing fibroblast cells in a basal medium;
collecting a conditioned medium;
providing avian gonadal cells with PGCs from sex glands; and
growing the PGCs in the conditioned medium supplemented with growth factors, wherein the conditioned medium provides the avian PGCs being culturing for a long period.

2. The method as claimed in claim 1, wherein the avian PGCs are fowl PGCs.

3. The method as claimed in claim 2, wherein the fowl PGCs are chicken PGCs.

4. The method as claimed in claim 1, wherein the fibroblast cells are inactivated or non-inactivated.

5. The method as claimed in claim 4, wherein the inactivated fibroblast cells are inactivated with mitomycin C.

6. The method as claimed in claim 1, wherein the fibroblast cells are chicken embryonic fibroblast cells or mouse embryonic fibroblast cells.

7. The method as claimed in claim 1, wherein the growth factors are leukocyte inhibition factor (LIF), stem cell factor (SCF), fibroblast growth factor, insulin growth factor-1, interleukin-11 or any combination of the foregoing factors.

8. The method as claimed in claim 1 further comprising treating the gonadal cells with an enzyme to form a suspension before growing the PGCs in the conditioned medium.

9. The method as claimed in claim 8, wherein the enzyme is trypsin.

10. The method as claimed in claim 8, wherein the suspension comprises PGCs and stroma cells.

11. The method as claimed in claim 9, wherein the suspension comprises PGCs and stroma cells.

12. The method as claimed in claim 9 further comprising:
centrifuging the suspension to remove the supernatant; and
resuspending the PGCs and stroma cells in the conditioned medium supplemented with the growth factors.

13. The method as claimed in claim 1, wherein the basal medium is Dulbecco's modified Eagle's medium (DMEM).

14. The method as claimed in claim 6, wherein the mouse embryonic fibroblast cells are mouse STO fibroblast cells.

15. A method for manufacturing a medium for culturing avian PGCs comprising:
providing a basal medium;
culturing fibroblast cells in the basal medium;
collecting a conditioned medium; and
adding growth factors into the cultured medium, wherein the conditioned medium provides the avian PGCs being culturing for a long period.

16. The method as claimed in claim 15, wherein the fibroblast cells are inactivated or non-inactivated.

17. The method as claimed in claim 15, wherein the fibroblast cells are chicken embryonic fibroblast cells or mouse embryonic fibroblast cells.

18. The method as claimed in claim 15, wherein the growth factors are leukocyte inhibition factor (LIF), stem cell factor (SCF), fibroblast growth factor, insulin growth factor-1, interleukin-11 or any combination of the foregoing factors.

19. The method as claimed in claim 15, wherein the basal medium is DMEM.

20. The method as claimed in claim 16, wherein the inactivated fibroblast cells are inactivated with mitomycin C.

21. A medium according to the method as claimed in claim 15 for culturing avian PGCs.
